(19) United States
(12) Patent Application Publication
(10) Pub. No.: US 2003/0149021 A1
(43) Pub. Date: Aug. 7, 2003

(54) Piperazine mono(dithio)carbamate ester compounds and analogs thereof: preparation method and uses thereof

(75) Inventors: Runtao Li, Beijing (CN); Tieming Cheng, Beijing (CN); Jingrong Cui, Beijing (CN); Tingmin Wang, San Marcos, CA (US)

Correspondence Address: FOLEY & LARDNER, P.O. BOX 80278, SAN DIEGO, CA, 92138-0278, US

(73) Assignee: Medinox, Inc.

(21) Appl. No.: 10/157,733

(22) Filed: May 28, 2002

(30) Foreign Application Priority Data
May 29, 2001 (CN) 01118399.3

(51) Int. Cl. C07D 279/12; C07D 265/32; A61K 31/55; A61K 31/553; A61K 31/554; A61K 31/551; A61K 31/54; A61K 31/537; A61K 31/495

(52) U.S. Cl. 514/211.01; 514/217.11; 514/218; 514/227.5; 514/255.01; 514/238.8; 514/317; 514/365; 514/374; 514/385; 540/544; 540/575; 544/59; 540/607; 546/226; 544/386; 544/170

(57) ABSTRACT

In accordance with the present invention, there is provided a new class of compounds, i.e., mono(dithio)carbamate esters of piperazine and analogs thereof, with or without substituents. Also provided are methods for the preparation of invention compounds and the pharmaceutical use thereof in the treatment of a variety of pathological conditions, especially for the treatment of cancers. A lead compound has shown good anticancer activity with low toxicity.

### CROSS-REFERENCE TO RELATED PATENT APPLICATIONS

This invention claims priority from Chinese Patent Application No. 01118399.3, filed May 29, 2001, now pending, the entire contents of which are hereby incorporated by reference herein.

### FIELD OF THE INVENTION

The present invention relates to novel mono(dithio)carbamate ester compounds of piperazine and analogs thereof, with or without substituents, or pharmaceutically acceptable salts thereof. The present invention also relates to methods for the preparation of invention compounds, as well as application thereof as pharmaceutically active compounds for treatment of a variety of pathological conditions, especially for treatment of cancers.

### BACKGROUND OF THE INVENTION

Dithiocarbamate ester compounds have been widely used in many areas, particularly in pesticides as antibacterial and antifungal agents. However, little research has been done about the physiological activity, e.g., the anticancer activity, of this class of compounds. Recently, Gerhauser (Cancer research, 57, 272-278(1997)) reported that a dithiocarbamate ester compound, Brassinium, separated from a plant of the mustard family, showed cancer preventive activity. A further study of similar compounds resulted in the discovery of Sulformate, a compound with improved cancer preventive activity.

In spite of these recent discoveries, the identification of additional compounds with desirable physiological activity would be of great interest in the field of medicine.

### SUMMARY OF THE INVENTION

In accordance with the present invention, there is provided a new class of compounds, i.e., mono(dithio)carbamate esters of piperazine and analogs thereof, with or without substituents. Also provided are methods for the preparation of invention compounds and the pharmaceutical use thereof in the treatment of a variety of pathological conditions, especially for the treatment of cancers. A lead compound has shown good anticancer activity with low toxicity.

### DETAILED DESCRIPTION OF THE INVENTION

In accordance with the present invention, there is provided a new class of compounds. Invention compounds have the structure:

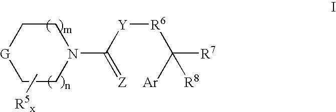

wherein:

G is a divalent radical selected from CR¹R², O, S(O)_{0.1, 2}, NR³ or N⁺R³R⁴ X⁻;

R¹ is H, OH, NO₂, COOH, CN, Cl, Br, I, keto (═O), thioketo (═S), imine (═NR, R is H, optionally substituted alkyl), an exocyclic double bond(═CH₂), —SO₃M or —OSO₃M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl, optionally substituted alkoxycarbonyl, alkylcarbonyloxy, optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino, optionally halogenated arylsulfonylamino, acyl, acyloxy, optionally substituted heterocyclic, a carbamate group, a sulfonamide group, sulfuryl, an amide group, or an optionally substituted ester group;

R² is absent when R¹ is keto, thioketo, imine, or an exocyclic double bond, or R² is H, OH, NO₂, COOH, CN, Cl, Br, I, —SO₃M or —OSO₃M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl, optionally substituted alkoxycarbonyl, alkylcarbonyloxy, optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino, optionally halogenated arylsulfonylamino, acyl, acyloxy, optionally substituted heterocyclic, a carbamate group, a sulfonamide group, sulfuryl, an amide group, or an optionally substituted ester group;

R³ is H, optionally substituted hydrocarbyl, optionally substituted heteroaryl, optionally substituted aroyl, optionally substituted acyl, optionally substituted heterocyclic, hydroxyl, optionally substituted alkoxy, optionally substituted alkylthio, amino, optionally substituted alkylamino, optionally substituted dialkylamino, optionally substituted organosulfonyl, optionally substituted organosulfinyl, an optionally substituted alkylsulfonate ester group, optionally substituted alkylamino carbonyl, optionally substituted alkylamino thiocarbonyl, an optionally substituted alkyl ester group, an optionally substituted alkyl thioester group, an optionally substituted sulfonamide group, an optionally substituted alkyl carbonate group, an optionally substituted alkyl thiocarbonate group or an optionally substituted phosphonate group;

R⁴ is optionally present when X⁻ is present; when present, R⁴ is H, optionally substituted hydrocarbyl, optionally substituted heteroaryl or optionally substituted heterocyclic;

R⁵ is optionally present, and when present each R⁵ is independently selected from OH, NO₂, COOH, CN, Cl, Br, I, keto (═O), thioketo (═S), imine (═NR, R is H, optionally substituted alkyl), an exocyclic double bond(═CH₂), —SO₃M or —OSO₃M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl, optionally substituted alkoxycarbonyl, alkylcarbonyloxy, optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino, optionally halogenated arylsulfonylamino, acyl, acyloxy, optionally substituted heterocyclic, a carbamate group, a sulfonamide group, sulfuryl, an amide group, or an optionally substituted ester group;

R⁶ is optionally substituted alkylene, hydrocarbylene, optionally substituted aminoalkylene, or optionally substituted heterocyclic;

R⁷ is H, CN, COOH, OH, Br, Cl, I, optionally substituted aryl, optionally substituted heteroaryl, or R⁷ and Ar can cooperate to form a macrocycle;

R⁸ is H, OH, NO₂, COOH, tetrazolyl, C(O)NHOH, CN, Cl, Br, I, —SO₃M, or OSO₂M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl; optionally substituted alkoxycarbonyl; alkylcarbonyloxy; optionally substituted heteroaryl, optionally substituted aroyl, optionally substituted arylcarbonyl; optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino or arylsulfonylamino, acyl, acyloxy, optionally substituted heterocyclic, a carbamate group, a sulfonamide group, sulfuryl, COOR⁹, COONR¹⁰R¹¹, wherein R⁹, R¹⁰ and R¹¹ are independently selected from C₁₋₁₀ alkyl;

Ar is optionally substituted aryl, optionally substituted heteroaryl, or Ar and R⁷ can cooperate to form a macrocycle;

X⁻ is present only when R⁴ is present; and when X⁻ is present, it is Cl⁻, Br⁻, I⁻, F⁻ nitrate, phosphate, sulfate, acetate, adipate, besylate, camyslate, citrate, edisylate, estolate, fumarate, gluceptate, gluconate, glucuronate, hippurate, hyclate, lactate, lactobionate, maleate, napsylate, oleate, pamoate, stearate, succinate, sulfosalicylate, tannate, tartrate, terephthalate, tosylate, or triethiodide;

m and n are independently integers from 0 up to 5;

x is an integer from 0 up to 4;

Y is a divalent radical selected from CR¹R², O or S; and

Z is O or S.

As employed herein, “alkyl” refers to saturated straight or branched chain hydrocarbyl radicals having 1 up to 20 carbon atoms, preferably 2-10 carbon atoms.

As employed herein, “substituted alkyl” comprises alkyl group further bearing one or more substituents selected from H, OH, NO₂, COOH, CN, Cl, Br, I, —SO₃M (M is H, Na, K, Zn, Ca, meglumine and the like), CSSH, COSH, CBr₂, CF₂, CF₃, CCl₂, CCl₃, OSO₃M (M is H, Na, K, Zn, Ca, meglumine and the like). C₁₋₂₀, preferably C₁₋₁₀ alkyl, substituted alkyl, alkenyl, substituted alkenyl, alkynyl, substituted alkynyl, cycloalkyl, substituted cycloalkyl, alkoxy, substituted alkoxy, alkylthio, substituted alkylthio, amino, monoalkylamino, dialkylamino, alkylcarbonyl, substituted alkylcarbonyl, alkoxycarbonyl, substituted alkoxycarbonyl, alkylcarbonyloxy, substituted alkylcarbonyloxy, aryl, substituted aryl (may be substituted by 1 to 5 substituents), heteroaryl, substituted heteroaryl, alkylaryl, substituted alkylaryl, arylalkyl, substituted arylalkyl, arylalkenyl, substituted arylalkenyl, arylalkynyl, substituted arylalkynyl, aroyl, substituted aroyl, arylcarbonyl, substituted arylcarbonyl, aryloxycarbonyl, substituted aryloxycarbonyl, arylcarbonyloxy, substituted arylcarbonyloxy, aralkyloxy, heteroarylcarbonyl, substituted heteroarylcarbonyl, optionally substituted arylthio, carbamoyl, monoalkylcarbamoyl, di-alkylcarbamoyl, arylcarbamoyl, alkylsulfinyl, alkylsulfonyl, aryloxy, optinally halogenated alkylsulfonylamino, and arylsulfonylamino, acyl group, acyloxy group, heterocyclic including heteroaryl group having one to four hetero atoms selected from the group consisting of a nitrogen atom, sulfur atom and oxygen atom, carbamate group, sulfonamide group, sulfuryl, amide group, ester groups, and the like.

As employed herein, “hydrocarbyl” refers to saturated or unsaturated, cyclic or acyclic radicals having in the range of about 1-20 carbon atoms, and composed of hydrogen and carbon only. Hydrocarbyl embraces alkyl, alkenyl, alkynyl, cycloalkyl, cycloalkenyl, aryl, alkylaryl, arylalkyl, aryalkenyl, alkenylaryl, arylalkynyl and alkynylaryl groups, and the like.

As employed herein, “cycloalkyl” refers to cyclic ring-containing groups containing in the range of 3 up to 20 carbon atoms, and “substituted cycloalkyl” refers to cycloalkyl groups further bearing one or more substitutents as set forth above.

As employed herein, “alkenyl” refers to straight or branched chainhydrocarbyl groups having at least one carbon-carbon double bond, and having in the range of 2 up to 20 carbon atoms, and “substituted alkenyl” refers to alkenyl groups further bearing one or more substitutents as set forth above.

As employed herein, “alkenyl” refers to straight or branched chainhydrocarbyl groups having at least one carbon-carbon triple bond, and having in the range of about 2 to 20 carbon atoms, and “substituted alkynyl” refers to alkynyl groups further bearing one or more substitutents as set forth above.

As employed herein, “aryl” refers to aromatic groups having in the range of 6 up to 20 carbon atoms and “substituted aryl” refers to aryl groups further bearing one or more substituents as set forth above.

As employed herein, “alkylaryl” refers to alkyl substituted aromatic groups and “substituted alkylaryl” refers to alkylaryl groups further bearing one or more substituents as set forth above.

As employed herein, “arylalkyl” refers to arylsubstituted alkyl groups and “substituted arylalkyl” refers to arylalkyl groups further bearing one or more substituents as set forth above.

As employed herein, “arylalkenyl” refers to arylsubstituted alkenyl groups and “substituted arylalkenyl” refers to arylalkenyl goups further bearing one or more substituents as set forth above.

As employed herein, “arylalkynyl” refers to aryl substituted alkynyl groups and “substituted arylalkynyl” refers to arylalkynyl groups further bearing one or more substituents as set forth above.

As employed herein, “hydrocarbylene” refers to divalent saturated or unsaturated, cyclic or acyclic radicals having in the range of about 1-20 carbon atoms, and composed of hydrogen and carbon only. Hydrocarbylene embraces alkylene, alkenylene, alkynylene, cycloalkylene, cycloalkenylene, arylene, alkylarylene, arylalkylene, arylalkenylene, alkenylarylene, arylalkynylene, and alkynylarylene groups, and the like.

As employed herein, “alkylene” refers to divalent hydrocarbyl radicals having 1 to 20 carbon atoms, preferably, 2-10 carbon atoms; and “substituted alkylene” refers to alkylene groups further bearing one or more substituents as set forth above.

As employed herein, “cycloalkylene” refers to cyclic-containing groups containing in the range of 3 up to 8 carbon atoms, and “substituted cycloalkylene” refers to cycloalkylene further bearing one or more substituents as set forth above.

As employed herein, “alkenylene” refers to straight or branched chain hydrocarbyl groups having at least one carbon-carbon double bond, and having the range of about 2 up to 20 carbon atoms, and “substituted alkenylene” refers to alkenylene groups further bearing one or more substituents as set forth above.

As employed herein, “alkynylene” refers to straight or branched chain hydrocarbyl groups having at least one carbon-carbon triple bond, and having the range of about 2 up to 20 carbon atoms, and “substituted alkynylene” refers to alkynylene groups further bearing one or more substituents as set forth above.

As employed herein, “arylene” refers to aromatic groups having in the range of 6 up to 20 carbon atoms and “substituted arylene” refers to arylene groups further bearing one or more substituents as set forth above.

As employed herein, “alkylarylene” refers to alkyl-substituted arylene groups and “substituted alkylarylene” refers to alkylarylene groups bearing one or more substituents as set forth above.

As employed herein, “arylalkylene” refers to aryl-substituted alkylene groups and “substituted arylalkylene” refers to arylalkylene groups bearing one or more substituents as set forth above.

As employed herein, “arylalkenylene” refers to aryl-substituted alkenylene groups and “substituted arylalkenylene” refers to arylalkenylene groups bearing one or more substituents as set forth above.

As employed herein, “arylalkynylene” refers to aryl-substituted alkynylene groups and “substituted arylalkynylene” refers to arylalkynylene groups bearing one or more substituents as set forth above.

As employed herein, “alkoxy” refers to —O-alkyl groups having in the range of 2 up to 20 carbon atoms and “substituted alkoxy” refers to alkoxy groups further bearing one or more substituents as set forth above.

As employed herein, “aryloxy” refers to —O-aryl groups having in the range of 6 up to 20 carbon atoms and “substituted aryloxy” refers to aryloxy groups further bearing one or more substituents as set forth above.

As employed herein, “alkylthio” refers to —S-alkyl groups having in the range of 2 up to 20 carbon atoms and “substituted alkylthio” refers to alkylthio groups further bearing one or more substituents as set forth above.

As employed herein, “amino” refers to the substituent —NH₂.

As employed herein, “monoalkylamino” refers to a substituent of the structure —NHR, wherein R is alkyl or substituted alkyl as set forth above.

As employed herein, “dialkylamino” refers to a substituent of the structure —NR₂, wherein each R is independently alkyl or substituted alkyl as set forth above.

As employed herein, “alkylcarbonyl” refers to —C(O)-alkyl groups having in the range of 2 up to 20 carbon atoms and “substituted alkylcarbonyl” refers to alkylcarbonyl groups further bearing one or more substituents as set forth above.

As employed herein, “alkylcarbonyloxy” refers to —OC(O)-alkyl groups having in the range of 2 up to 20 carbon atoms and “substituted alkylcarbonyloxy” refers to alkylcarbonyloxy groups further bearing one or more substituents as set forth above.

As employed herein, “alkyloxycarbonyl” refers to —C(O)O-alkyl groups having in the range of 2 up to 20 carbon atoms and “substituted alkyloxycarbonyl” refers to alkyloxycarbonyl groups further bearing one or more substituents as set forth above.

As employed herein, “arylcarbonyl” refers to —C(O)-aryl groups having in the range of 7 up to 21 carbon atoms and “substituted arylcarbonyl” refers to arylcarbonyl groups further bearing one or more substituents as set forth above.

As employed herein, “aryloxycarbonyl” refers to —C(O)Q-aryl groups having in the range of 7 up to 21 carbon atoms and “substituted aryloxycarbonyl” refers to aryloxycarbonyl groups further bearing one or more substituents as set forth above.

As employed herein, “arylcarbonyloxy” refers to —OC(O)-aryl groups having in the range of 7 up to 21 carbon atoms and “substituted arylcarbonyloxy” refers to arylcarbonyloxy groups further bearing one or more substituents as set forth above.

As employed herein, “aralkyloxy” refers to —O-arylalkyl groups and “substitiuted aralkyloxy” refers to aralkyloxy groups further bearing one or more substituents as set forth above.

As employed herein, “arylthio” refers to —S-aryl groups having in the range of 6 up to 20 carbon atoms and “substituted arylthio” refers to alkylthio groups further bearing one or more substituents as set forth above.

As employed herein, “aroyl” refers to arylcarbonyl species such as benzoyl and “substituted aroyl” refers to aroyl groups further bearing one or more substituents as set forth above.

As employed herein, “heterocyclic” refers to cyclic (i.e. ring containing) groups containing one or more heteroatoms (e.g. N, O, S, or the like) as part of the ring structure, and having in the range of 3 up to 20 carbon atoms, and “substituted heterocyclic” refers to heterocyclic groups further bearing one or more substituents as set forth above.

As employed herein, “heteroaryl” refers to aromatic (i.e. ring containing) groups containing one or more heteroatoms (e.g. N, O, S, or the like) as part of the ring structure, and having in the range of 4 up to 20 carbon atoms, and “substituted heteroaryl” refers to heteroaryl groups further bearing one or more substituents as set forth above.

As employed herein, “heteroarylcarbonyl” refers to the moiety —C(O)-heteroaryl, wherein heteroaryl is as defined above, and “substituted heteroarylcarbonyl” refers to heteroarylcarbonyl groups further bearing one or more substituents as set forth above.

As employed herein, “acyl” refers to alkylcarbonyl species (i.e., alkyl-C(O)—) and “substituted acyl” refers to acyl groups bearing one or more substituents as set forth above.

As employed herein, “acyloxy” refers to the moiety —O-acyl, wherein acyl is as defined above, and “substituted acyloxy” refers to acyloxy groups further bearing one or more substituents as set forth above.

As employed herein, “halogen” refers to fluoride, chloride, bromide or iodide atoms.

As employed herein, “oxyalkylene” refers to the moiety —O-alkenylene-, wherein alkylene is as defined above, and the “substituted oxyalkylene” refers to oxyalkylene groups further bearing one or more substituents as set forth above.

As employed herein, reference to “an amide group” embraces substituents of the structure —C(O)—NR₂, wherein each R is independently H, alkyl, substituted alkyl, aryl or substituted aryl as set forth above. When each R is H, the substituent is also referred to as “carbamoyl” (i.e., a substituent having the structure —C(O)—NH₂). When only one of the R groups is H, the substituent is also referred to as “monoalkylcarbamoyl” (i.e., a substituent having the structure —C(O)—NHR, wherein R is alkyl or substituted alkyl as set forth above) or “arylcarbamoyl” (i.e., a substituent having the structure —C(O)—NH(aryl), wherein aryl is as defined above, including substituted aryl). When neither of the R groups are H, the substituent is also referred to as “di-alkylcarbamoyl” (i.e., a substituent having the structure —C(O)—NR₂, wherein each R is independently alkyl or substituted alkyl as set forth above).

As employed herein, “organosulfinyl” refers to substituents having the structure —S(O)-organo, wherein organo embraces alkyl-, alkoxy- and alkylamino-moieties, as well as substituted alkyl-, alkoxy- or alkylamino-moieties.

As employed herein, “organosulfonyl” refers to substituents having the structure —S(O)₂-organo, wherein organo embraces alkyl-, alkoxy- and alkylamino-moieties, as well as substituted alkyl-, alkoxy- or alkylamino-moieties.

As employed herein, “optionally halogenated alkylsulfonylamino” refers to substituents of the structure —NH—S(O)₂-alkyl, wherein alkyl is as defined above, including substituted alkyl.

As employed herein, “optionally halogenated arylsulfonylamino” refers to substituents of the structure —NH—S(O)₂-aryl, wherein aryl is as defined above, including substituted aryl.

As employed herein, reference to “a carbamate group” embraces substituents of the structure —O—C(O)—NR₂, wherein each R is independently H, alkyl, substituted alkyl, aryl or substituted aryl as set forth above.

As employed herein, reference to “a sulfonamide group” embraces substituents of the structure —S(O)₂—NH₂.

As employed herein, “sulfuryl” refers to substituents of the structure ═S(O)₂.

As employed herein, reference to “an ester group” embraces substituents of the structure —O—C(O)-hydrocarbyl or —C(O)—O-hydrocarbyl, wherein hydrocarbyl is as defined above, including substituted hydrocarbyl.

In accordance with one aspect of the present invention, preferred compounds contemplated by the present invention include those of Formula I as set forth herein, wherein:

G is N⁺R³R⁴ X⁻;

R³ is H, optionally substituted C₁₋₁₀ alkyl, or optionally substituted aryl;

R⁴ is present only if X⁻ is present, and when present R⁴ is H, or C₁₋₁₀ alkyl;

each R⁵, if present, is independently OH, NO₂, COOH, ═O, ═S, CN, Cl, Br, or optionally substituted lower alkyl;

R⁶ is an optionally substituted C₁₋₅ alkylene;

R⁷ is H, CN, COOH, optionally substituted aryl or optionally substituted heteroaryl;

R⁸ is H, OH, NO₂, COOH, tetrazolyl, CN, Cl, Br, I, or lower alkyl;

Ar is an optionally substituted aryl;

X⁻ is present only when R⁴ is present; and when X⁻ is present, it is Cl⁻, Br⁻, I⁻ or F⁻;

m and n are independently integers from 1 to 3;

x is an integer from 0 to 2; and

Y and Z are independently O or S.

In accordance with another aspect of the present invention, preferred compounds contemplated by the present invention include those of Formula I as set forth herein, wherein:

G is N⁺R³R⁴ X⁻;

R³ is H, or an optionally substituted lower alkyl;

R⁴, if present, is H;

each R⁵ if present, is independently OH, CN, ═O, COOH or lower alkyl;

R⁶ is a C₁₋₃ alkylene;

R⁷ is H, COOH, CN, or optionally substituted aryl;

R⁸ is H, OH, COOH, CN or tetrazolyl;

Ar is an optionally substituted phenyl;

X⁻, if present, is Cl⁻;

m and n are both 1;

x is 0 or 1; and

Y and Z are S.

In accordance with yet another aspect of the present invention, preferred compounds contemplated by the present invention include those of Formula I as set forth herein, wherein:

G is N⁺R³R⁴ X⁻;

R³ is an optionally substituted C₁₋₂ alkyl;

R⁴ is H;

R⁵ is not present:

R⁶ is ethylene;

R⁷ is an optionally substituted phenyl;

R⁸is COOH or CN;

Ar is an optionally substituted phenyl;

X⁻ is Cl⁻;

m and n are both 1;

x is 0; and

Y and Z are S.

A presently preferred compound according to the invention has the structure referred to as compound 22, as follows:

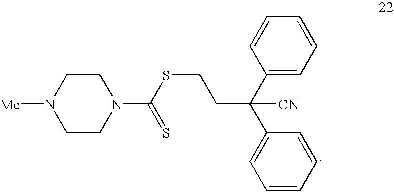

Another presently preferred compound according to the invention has the structure referred to as compound 42, as follows:

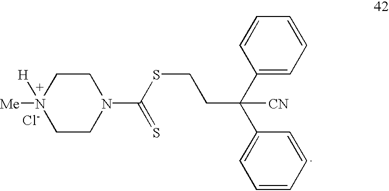

Diseases and conditions contemplated for treatment in accordance with the present invention include inflammatory and infectious diseases, such as, for example, septic shock, hemorrhagic shock, anaphylactic shock, toxic shock syndrome, ischemia, cerebral ischemia, administration of cytokines, overexpression of cytokines, ulcers, inflammatory bowel disease (e.g. ulcerative colitis or Crohn's disease), diabetes, arthritis (e.g. rheumatoid arthritis and osteoarthritis), asthma, Alzheimer's disease, Parkinson's disease, multiple sclerosis, cirrhosis, allograft rejection, encephalomyelitis, meningitis, pancreatitis, vasculitis, lymphocytic choriomeningitis, glomerulonephritis, uveitis, ileitis, inflammation (e.g. liver inflammation, renal inflammation, and the like), burn, infection (including bacterial, viral, fungal, and parastic infections), hemodialysis, chronic fatigue syndrome, stroke, cancers (e.g. breast, melanoma, carcinoma, and the like), cardiopulmonary bypass, ischemic/reperfusion injury, gastritis, adult respiratory distress syndrome, cachexia, myocarditis, autoimmune disorder, eczema, psoriasis, heart failure, heart disease, atherosclerosis, dermatitis, urticaria, systemic lupus erythematosus, AIDS, AIDS dementia, chronic neurodegenerative disease, pain (e.g. chronic pain and post-surgical pain), priapism, cystic fibrosis, amyotrophic lateral sclerosis, schizophrenia, depression, premenstrual syndrome, anxiety, addiction, migraine, headache, Huntington's disease, epilepsy, neurodegenerative disorders, gastrointestinal motility disorders, obesity, hyperphagia, solid tumors, malaria, hematologic cancers, myelofibrousis, lung injury, graft-versus-host disease, head injury, CNS trauma, hepatitis, renal failure, liver disease, drug-induced lung injury, myasthenia gravis (MG), ophthalmic disease, post-angioplasty, restenosis, anginia, coronary artery disease, and the like.

Presently preferred diseases and conditions contemplated for treatment in accordance with the present invention are cancers and tumors.

In accordance with further embodiment of the present invention, there are provided physiologically active composition(s) comprising invention compounds in a suitable vehicle rendering invention compounds amenable to oral delivery, transdernal delivery, intravenous delivery, intramuscularly delivery, topical delivery, nasal delivery, and the like.

Pharmaceutical compositions of the present invention can be used in the form of a solid, a solution, and emulsion, a dispersion, a micelle, a liposome, and the like, wherein the resulting composition contains one or more of the compounds of the present invention, as an active ingredient, in admixture with an organic or inorganic carrier or excipient suitable for enterable or parenteral applications. The active ingredient may be compounded, for example, with the usual non-toxic, pharmaceutically acceptable carriers for tablets, pellets, capsules, suppositories, solutions, emulsions, suspensions and any other suitable for use. The carriers which can be used include glucose, lactose, gum acacia, gelatin, manitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening, and coloring agents and perfumes may be used. The active compound(s) is (are) included in the pharmaceutically active composition in an amount sufficient to produce the desired effect upon the process or disease condition.

Pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in or to provide pharmaceutically elegant and palatable preparations. Tablets containing the active ingredient in admixture with non-toxic pharmaceutically acceptable excipients used may be, for example, (1) inert diluents such as calcium carbonate, lactose, calcium phosphate or sodium phosphate; (2) granulating and disintegrating agents such corn starch, potato starch or alginic acid; (3) binding agents such as gum tragacanth, corn starch, gelatin or acacia, and (4) lubricating agents such as maganesium stearate, steric acid or talc. The tablets may be uncoated or they may be coated by known techniques to delay disintegration and absorption in the gastrointestinal tract and thereby provide a sustained action over a longer period. For example, a time delaymaterial such as glyceryl monostearate or glyceryl distearate may be employed. They may also be coated by the techniques described in the U.S. Pat Nos. 4,256,108; 4,160,452; and 4,265,874, to form osmotic therapeutic tablets for controlled release.

In some cases, formulations for oral use may be in the form of hard gelatin capsules wherein the active ingredients is mixed-with an inert solid diluents, for example, calcium carbonate, calcium phosphate or kaolin. They may also be in the form of soft gelatin capsules wherein the active ingredient is mixed with water or an oil medium, for example, peanut oil, liquid paraffin, or olive oil.

The pharmaceutical compositions may be in the form of a sterile injectable suspension. This suspension may be formulated according to known methods using suitable dispersing or wetting agents and suspending agents. The sterile injectable preparation may also be a sterile injectable solution or suspension in a non-toxic parenterally-acceptable diluents or solvent, for example, as a solution in 1,3-butanediol. Sterile, fixed oils are conventionally employed as a solvent or suspending medium. For this purpose any bland fixed oil may be employed including synthetic mono- or diglycerides, fatty acids, naturally occurring vegetable oils like sesame oil, coconut oil, penult oil, cottonseed oil, etc., or synthetic fatty vehicles like ethyl oleate or the like. Buffers, preservatives, antioxidants, and the like can be incorporated as required.

Compounds contemplated for use in the practice of the present invention may also be administered in the form of suppositories for rectal administration of the drug. These compositions may be prepared by mixing the drug with a suitable non-irritating excipient, such as cocoa butter, synthetic glyceride esters of polyethylene glycols, which are solid at ordinary temperatures, but liquefy and/or dissolve in the rectal cavity to release the drug. Since individual subjects may present a wide variation in severity of symptoms and each drug has its unique therapeutic characteristics, the precise mode of administration and dosage employed for each subject is left to the discretion of the practitioner.

In accordance with yet another embodiment of the present invention, there are provided improved methods for the treatment of a subject suffering from a pathological condition by administration thereto of pharmaceutically active agent(s). Thus, the invention method for the treatment of a subject afflicted with a pathological condition comprises administering to a subject in need thereof an effective amount of a pharmaceutically active agent.

In accordance with yet another embodiment of the present invention, said pathological condition is at least one cancer and/or tumor.

In accordance with still another embodiment of the present invention, there are provided methods for the preparation of the invention compounds, said method comprising the following. As readily recognized by those of skill in the art, invention compounds can be prepared in a variety of ways.

General Preparation Method of a Starting Material (Scheme 1)

One of the starting materials with structure CHR⁷ArCN if not commercially available can be prepared as follows:

1. Compound II (e.g., acetonitrile, aromaticacetonitrile, substituted aromaticacetonitrile, heteroaromaticacetonitrile or substituted heteroaromaticacetonitrile, or the like) is brominated to give the corresponding bromo compounds (III).

2. Friedel-Crafts reaction of the above compounds with aromatic or heteroaromatic compounds in the presence of Lewis acid generates the corresponding compounds CHR⁷ArCN (IV).

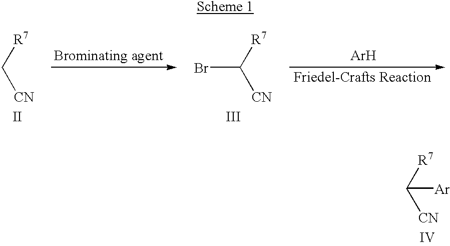

General Procedure for the Preparation of Invention Compounds (Schemes 2-4)

A. Reaction of CHR⁷R⁸Ar(V) with Br—R⁶—Br (α,ω-Br,Br—R⁶) gives a compound BrR⁶CR⁷R⁸Ar (VI), wherein R⁶, R⁷, R⁸ and Ar are as defined above

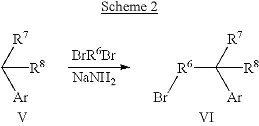

B. Reaction of compound VI with 1-substituted (R¹) piperazine (VII) and carbon disulfide (CS₂) in the presence of potassium phosphate in acetone or dimethylformamide as a solvent generates compound (VIII).

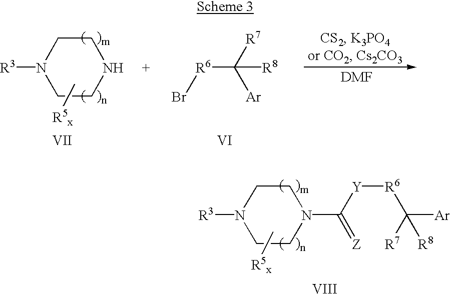

wherein

R³, R⁵, R⁶, R⁷, R⁸, Ar, m, n, x, Y and Z are as defined above.

C. Compound VIII is treated with alkyl halide, organic or inorganic acids generates the corresponding salts (IX)

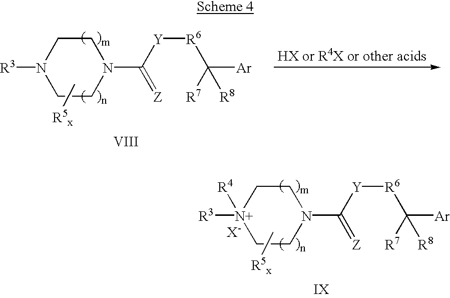

wherein

R³, R⁴, R⁵, R⁶, R⁷, R⁸, Ar, X⁻, m, n, x, Y and Z are as defined above.

D. As readily recognized by those of skill in the art, invention compound X can be converted to the corresponding acid (XI), the corresponding ester (XII), amide (XIII) or tetrazolyl (XIV) derivatives, as illustrated below:

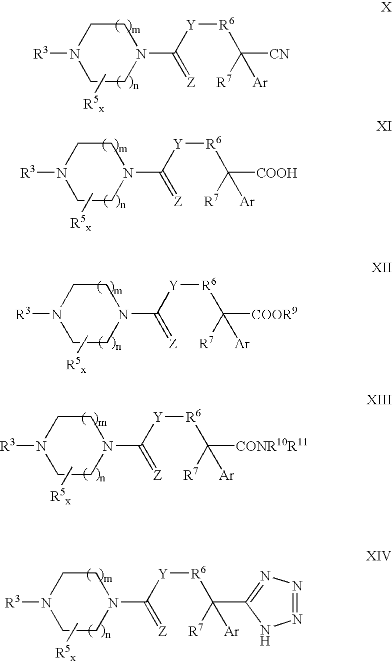

wherein

R³, R⁵, R⁶, R⁷, R⁸, R⁹, R¹⁰, R¹¹, Ar, X⁻, m, n, x, Y and Z are as defined above.

Furthermore, the invention compounds can be used in the field of pesticide as insecticide, antibacterial agents either alone or as an active ingredient of a composition.

The invention will now be described in greater detail by reference to the following non-limiting examples.

### EXAMPLES

The syntheses described in examples 1-20 are outlined in Scheme 5.

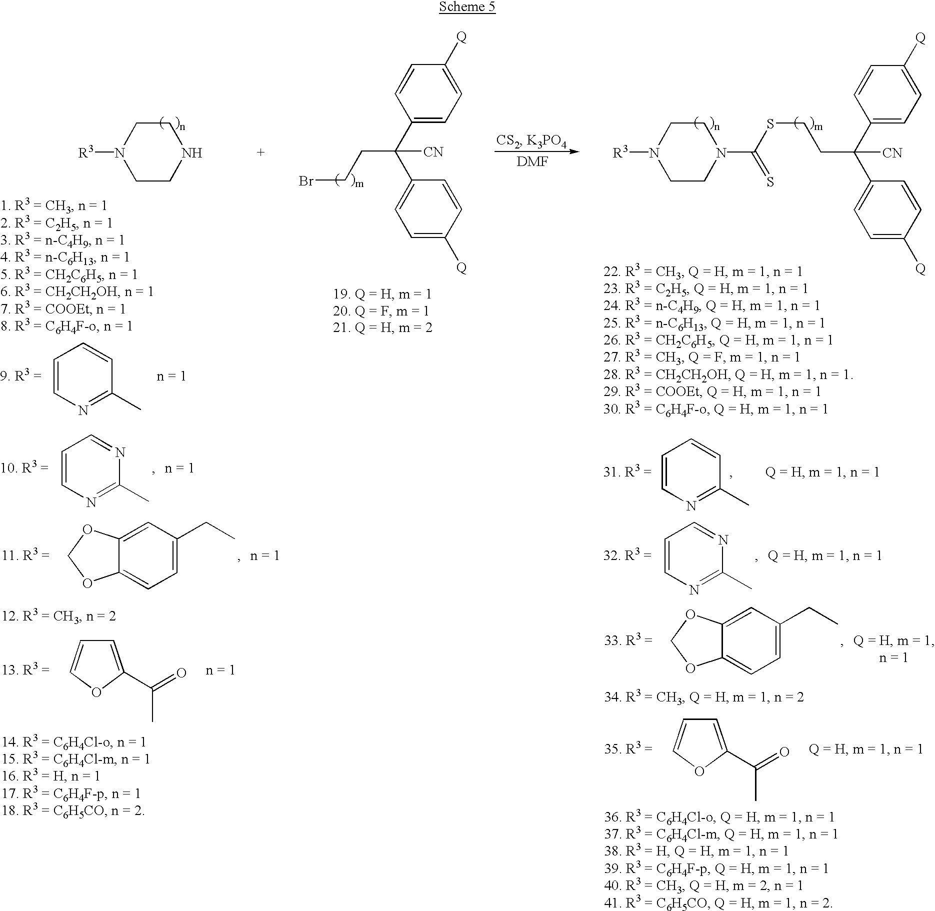

### Example 1

### 4-Methyl-piperazine-1-carbodithioc acid 3-cyano-3,3-diphenyl-propyl ester (22) (Scheme 5)

To a stirring mixture of 1-Methylpiperazine (1) (10.7 g, 0.1 mol), anhydrous potassium phosphate (21.2 g, 0.1 mol) in acetone (250 ml) was added carbon disulfide (9.1 g, 0.12 mol) at room temperature (rt). The mixture was stirred at rt for 30 min. 2,2-Diphenyl-4-bromobutyronitrile (19) (30.2 g, 0.1 mol) was added and the resulting mixture was continued to stir for additional one hour. The reaction mixture was filtered and the solvent was evaporated. The residue was dissolved in ethyl acetate (250 ml). The solution was washed with water and dried (CaCl₂). After partial evaporation of the solvent, a crystal was obtained. The compound was purified by recrystallization from ethanol to give 24 g (60%) of compound 22 as a white crystal: mp. 110-112° C.; ¹H NMR (CDCl₃) δ2.31 (s, 3H), 2.47 (br, 4H), 2.82 (m, 2H), 3.37 (m, 2H), 3.89 (br, 2H), 4.32 (br, 2H), 7.30 (t, 2H, J=7.5 Hz), 7.37 (t, 4H, J=7.5 Hz), 7.47 (d, 4H, J=7.5 Hz); ¹³C NMR (CDCl₃) δ, 32.57, 39.50, 45.77, 49.96, 51.31, 51.80, 54.51 121.14, 127.18, 128.25, 129,10, 139.44, 196.10; Anal. Calcd. For C₂₂H₂₂N₃S₂ C 66.80; H 6.37; N 10.62; S 16.21; Found: C 66.43; H 6.24; N 10.35; S 16.01.

### Example 2

### 4-Ethyl-piperazine-1-carbodithioc acid 3-cyano-3,3-diphenyl-propyl ester (23) (Scheme 5)

Compound 23 was synthesized as described above for the preparation of compound 22, this time employing 1-ethylpiperazine (2) and compound 19. mp. 118-120° C.; ¹H NMR (CDCl₃) δ1.12 (t, 3H, J=7.2 Hz), 2.54 (br, 6H), 2.83 (m, 2H), 3.38 (dd, 2H), 3.94 (br, 2H), 4.34 (br, 2H), 7.26-7.49 (m, 10H); Anal. Calcd. For C₂₃H₂₇N₃S₂, C 67.44; H 6.64; N 10.26; S 15.66; Found: C 67.32; H 6.37; N 10.43; S 15.91.

### Example 3

### 4-Butyl-piperazine-1-carbodithioc acid 3-cyano-3,3-diphenyl-propyl ester (24) (Scheme 5)

Compound 24 was synthesized as described above for the preparation of compound 22, this time employing 1-butylpiperazine (3) and compound 19. mp. 88-90° C.; ¹H NMR (CDCl₃) δ0.93 (t, 3H, J=7.8 Hz), 1.35 (m, 2H), 1.49 (m, 2H), 2.38 (br, 6H), 2.52 (br, 4H), 2.83 (m, 2H), 3.38 (dd, 2H), 3.92 (br, 2H), 4.34 (br, 2H), 7.26-7.49 (m, 10H); Anal. Calcd. For C₂₅H₃₁N₃S₂, C 68.61; H 7.14; N 9.60; S 14.65; Found: C 68.36; H 7.37; N 9.56; S 14.93.

### Example 4

### 4-Hexanyl-piperazine-1-carbodithioc acid 3-cyano-3,3-diphenyl-propyl ester (25) (Scheme 5)

Compound 25 was synthesized as described above for the preparation of compound 22, this time employing 1-hexanylpiperazine (4) and compound 19. mp. 53-55° C.; ¹H NMR (CDCl₃) δ0.88 (t, 3H, J=7.2 Hz), 1.29 (br, 6H), 1.49 (br, 2H), 2.37 (br, 2H), 2.52 (br, 4H), 2.83 (m, 2H), 3.38 (dd, 2H), 3.94 (br, 2H), 4.32 (br, 2H), 7.26-7.49 (m, 10H); Anal. Calcd. For C₂₇H₃₅N₃S₂, C 69.63; H 7.57; N 9.02; S 13.77; Found: C 69.34; H 7.26; N 9.12; S 13.87.

### Example 5

### 4-Benzyl-piperazine-1-carbodithioc acid 3-cyano-3,3-diphenyl-propyl ester (26) (Scheme 5)

Compound 26 was synthesized as described above for the preparation of compound 22, this time employing 1-benzylpiperazine (5) and compound 19; mp. 106-108° C.; ¹H NMR (CDCl₃) δ2.52 (br, 4H), 2.82 (m, 2H), 3.37 (dd, 2H), 3.54 (s, 2H), 3.88 (br, 2H), 4.31 (br, 2H), 7.26-7.49 (m, 15H); Anal. Calcd. For C₂₈H₂₉N₃S₂, C 71.30; H 6.20; N 8.91; S 13.60; Found: C 71.37; H 6.13; N 9.07; S 13.42.

### Example 6

### 4-Methyl-piperazine-1-carbodithioc acid 3-cyano-3,3-di(4-florophenyl)-propyl ester (27) (Scheme 5)

Compound 27 is synthesized as described above for the preparation of compound 22, this time employing 2,2- di(4-florophenyl)-4-bromobutyronitrile (20) and compound 1.

### Example 7

### 4-(2-Hydroxyethyl)-piperazine-1-carbodithioc acid 3-cyano-3,3-diphenyl-propyl ester (28) (Scheme 5)

Compound 28 was synthesized as described above for the preparation of compound 22, this time employing compound 6 and compound 19. ¹H NMR (CDCl₃) δ2.58 (t, 6H, J=5.0 Hz), 2.82 (m, 2H), 3.38 (m, 2H), 3.65 (t, 2H, J=5.5 Hz ), 3.92 (br, 2H), 4.33 (br, 2H), 7.30 (dd, 2H, J=8.5 Hz and 5.5 Hz), 7.37 (dd, 4H, J=8.5 Hz and 5.5 Hz), 7.46 (d, 4H, J=8.4 Hz); MS (ESI) m/z 426.6 (M +1)⁺.

### Example 8

### 4-Ethoxycarbonyl-piperazine-1-carbodithioc acid, (3-cyano-3,3-diphenyl)-propyl ester (29) (Scheme 5)

Compound 29 was synthesized as described above for the preparation of compound 22, this time employing compound 7 and compound 19. ¹H NMR (CDCl₃) δ1.28(t, 3H, J=7.0 Hz), 2.82(m, 2H), 3.39 (m, 2H), 3.58 (t, 4H, J=5.0 Hz), 3.91 (br, 2H), 4.17 (q, 2H, J=7.0 Hz), 4.35 (br, 2H), 7.30 (t, 2H, J=7.0 Hz), 7.37 (t, 4H, J=7.0 Hz), 7.46 (d, 4H, J=7.0 Hz); MS (ESI) m/z 454.6 (M+1)⁺.

### Example 9

### 4-(2-Fluoro-phenyl)-piperazine-1-carbodithioc acid 3-cyano-3,3-diphenyl-propyl ester (30) (Scheme 5)

Compound 30 was synthesized as described above for the preparation of compound 22, this time employing compound 8 and compound 19. ¹H NMR (CDCl₃) δ2.85 (m, 2H), 3.16 (t, 4H, J=5.0 Hz), 3.41 (m, 2H), 4.08 (br, 2H), 4.49 (br, 2H), 6.91 (m, 1H), 7.00 (m, 1H), 7.06 (m, 2H), 7.31 (t, 2H, J=7.5 Hz), 7.38 (t, 4H, J=7.5 Hz), 7.48 (d, 4H, J=7.8 Hz); MS (ESI) m/z 476.3 (M+1)⁺.

### Example 10

### 4-(2-Pyridyl)-piperazine-1-carbodithioc acid 3-cyano-3,3-diphenyl-propyl ester (31) (Scheme 5)

Compound 31 was synthesized as described above for the preparation of compound 22, this time employing compound 9 and compound 19. ¹H NMR (CDCl₃) δ2.85 (m, 2H), 3.41 (m, 2H), 3.70 (m, 4H), 4.03 (br, 2H), 4.44 (br, 2H), 6.62 (d, 1H, J=7.1 Hz), 6.68 (dd, 1H, J=6.8 Hz and 5.0 Hz), 7.31 (t, 2H, J=7.5 Hz), 7.37 (m, 4H, J=7.5 Hz), 7.47 (d, 4H, J=7.8 Hz), 7.52 (m, 1H), 8.20 (dd, 1H, J=5.0 and 1.5 Hz); MS (ESI) m/z 459.4 (M+1)⁺.

### Example 11

### 4-(2-Pyrimidyl)-piperazine-1-carbodithioc acid 3-cyano-3,3-diphenyl-propyl ester (32) (Scheme 5)

Compound 32 was synthesized as described above for the preparation of compound 22, this time employing compound 10 and compound 19. ¹H NMR (CDCl₃) δ2.85 (m, 2H), 3.42 (m, 2H), 3.94 (m, 4H), 3.95 (br, 2H), 4.40 (br, 2H), 6.56 (t, 1H, J=5.0 Hz), 7.31 (t, 2H, J=7.5 Hz), 7.38 (m, 4H), 7.48 (d, 4H, J=8.4 Hz); MS (ESI) m/z 460.5 (M+1)⁺.

### Example 12

### 4-Piperonyl-piperazine-1-carbodithioc acid 3-cyano-3,3-diphenyl-propyl ester (33) (Scheme 5)

Compound 33 was synthesized as described above for the preparation of compound 22, this time employing compound 11 and compound 19. ¹H NMR (CDCl₃) δ2.49 (br, 4H), 2.82 (m, 2H), 3.38 (m, 2H), 3.44 (s, 2H), 3.88 (br, 2H), 4.31 (br, 2H), 5.95 (s, 2H), 6.74 (m, 2H), 6.84 (s, 1H), 7.30 (t, 2H, J=7.5 Hz), 7.37 (t, 4H, J=7.5 Hz), 7.47 (d, 4H, J=7.5 Hz); ¹³C NMR (CDCl₃) δ32.61, 39.58, 50.18, 51.47, 51.86, 52.41, 62.40, 101.20, 108.18, 109.55, 122.11, 122.45, 127.45, 128.30, 129.14, 131.41, 139.50, 147.10, 148.00, 195.94; MS (ESI) m/z 516.7 (M+1)⁺.

### Example 13

### 4-Methyl-homopiperazine-1-carbodithioc acid 3-cyano-3,3-diphenyl-propyl ester (34) (Scheme 5)

Compound 34 was synthesized as described above for the preparation of compound 22, this time employing compound 12 and compound 19. ¹H NMR (CDCl₃) δ2.30 (br, 2H), 2.36 (d, 3H, J=2.5 Hz), 2.55 (t, 2H, J=5.5 Hz), 2.71 (m, 2H), 2.84 (m, 2H), 3.37 (m, 2H), 3.89 (t, 1H, J=6.0 Hz), 3.95 (t, 1H, J=5.0 Hz), 4.21 (t, 1H, J=6.0 Hz), 4.35 (m, 1H), 7.30 (t, 2H, J=7.0 Hz), 7.37 (t, 4H, J=7.5 Hz), 7.47 (d, 4H, J=7.5 Hz); MS (ESI) m/z 410.8(M+1)⁺.

### Example 14

### 4-Furoylpiperazine-1-carbodithioic acid 3,3-diphenyl-propyl ester (35) (Scheme 5)

Compound 35 was prepared as described above for the preparation of compound 22, this time employing compound 13 and compound 19. ¹H NMR (CDCl₃) δ2.84 (m, 2H), 3.40 (m, 2H), 3.94 (br, 6H), 4.40 (br, 2H), 6.51 (t, 1H), 7.09 (d, 1H), 7.31 (t, 2H), 7.37 (t, 4H), 7.46 (d, 4H), 7.51 (s, 1H); MS (ESI) m/z 498.6 (M+Na).

### Example 15

### 4-(2-Chloro-phenyl)-piperazine-1-carbodithioic acid 3-cyano-3,3-diphenyl-propyl ester (36) (Scheme 5)

Compound 36 was prepared as described above for the preparation of compound 22, this time employing compound 14 and compound 19. ¹H NMR (CDCl₃) δ2.86 (m, 2H), 3.12 (br, 4H), 3.41 (m, 2H), 4.08 (br, 2H), 4.50 (br, 2H), 7.01 (m, 2H), 7.26 (m, 2H), 7.31 (m, 2H), 7.36 (m, 4H), 7.47 (d, 4H, J=8.0 Hz); MS (ESI) m/z 493.0 (M+1)⁺.

### Example 16

### 4-(3-Chloro-phenyl)-piperazine-1-carbodithioic acid 3-cyano-3,3-diphenyl-propyl ester (37) (Scheme 5)

Compound 37 was prepared as described above for the preparation of compound 22, this time employing compound 15 and compound 19. ¹H NMR (CDCl₃) δ2.85 (m, 2H), 3.30 (br, 4H), 3.41 (m, 2H), 4.06 (br, 2H), 4.45 (br, 2H), 6.77 (dd, 1H, J=1.9 and 7.5 Hz), 6.86 (d, 2H, J=7.5 Hz), 7.19 (t, 1H, J=8.0 Hz), 7.31(t, 2H, J=7.5 Hz), 7.38 (t, 4H, J=7.5 Hz), 7.47 (d, 4H, J=8.0 Hz); MS (ESI) m/z 493.0 (M+1)⁺.

### Example 17

### Piperazine-1-carbodithioic acid 3-cyano-3,3-diphenyl-propyl ester (38) (Scheme 5)

Compound 38 was prepared as described above for the preparation of compound 22, this time employing compound 16 and compound 19. ¹H NMR (CDCl₃) δ1.72 (br, 1H), 2.83 (m, 2H), 2.93 (br, 4H), 3.39 (m, 2H), 3.87 (br, 2H), 4.29 (br, 2H), 7.30 (t, 2H), 7.38 (t, 4H), 7.46 (d, 4H); MS (ESI) m/z 382.4 (M+1)⁺.

### Example 18

### 4-(4-Fluoro-phenyl)-piperazine-1-carbodithioic acid 3-cyano-3,3-diphenyl-propyl ester (39) (Scheme 5)

Compound 39 was prepared as described above for the preparation of compound 22, this time employing compound 17 and compound 19. ¹H NMR (CDCl₃) δ2.85 (m, 2H), 3.19 (t, 4H), 3.41 (m, 2H), 4.07 (br, 2H), 4.46 (br, 2H), 6.87 (m, 2H), 6.99 (m, 2H), 7.31 (t, 2H), 7.48 (d, 4H); MS (ESI) m/z 476.5 (M+1)⁺.

### Example 19

### 4-Methylpiperazine-1-carbodithioc acid 4-cyano-4,4-diphenyl-butyl ester (40) (Scheme 5)

Compound 40 was prepared as described above for the preparation of compound 22, this time employing compound 21 and compound 1. ¹H NMR (CDCl₃) δ1.86 (m, 2H), 2.32 (s, 3H), 2.47 (br, 4H), 2.52 (m, 2H), 3.37 (t, 2H, J=7.2 Hz), 3.93 (br, 2H), 4.33 (br, 2H), 7.20 (m, 2H), 7.30 (m, 2H), 7.38 (m, 6H); MS (ESI) m/z 410.7 (M+1)⁺.

### Example 20

### 4-Benzoyl-homopiperazine-1-carbodithioc acid 4-cyano-4,4-diphenyl-propyl ester (41) (Scheme 5)

Compound 41 was prepared as described above for the preparation of compound 22, this time employing compound 19 and compound 18. mp. 56-58° C.; ¹H NMR (CDCl₃) δ1.88-2.17 (m, 2H), 2.81-2.86 (m, 2H), 3.37-4.27 (m, 10H), 7.22-7.48 (m, 15H); Anal. Calcd. For C₂₉H₂₇N₃S₂, C 69.71; H 5.85; N 8.41; Found: C 69.47; H 5.77; N 8.21; MS (FAB) m/z 499.9 (M⁺).

The syntheses described in Examples 21 & 22 are outlined in Scheme 6.

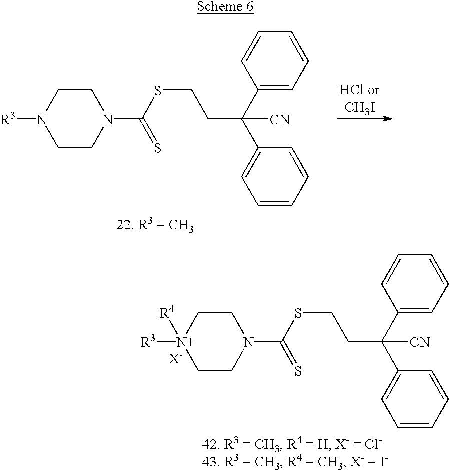

### Example 21

### 4-Methyl-piperazine-1-carbodithioc acid 3-cyano-3,3-diphenyl-propyl ester hydrochloride (42) (Scheme 6)

Compound 22 was treated with hydrochloric acid in ethanol to give compound 42 as a white crystal. mp>180° C. (dec); ¹H NMR (DMSO-d₆) δ2.75 (s, 3H), 2.84 (m, 2H), 3.11 (br, 2H), 3.22 (m, 2H), 3.44 (br, 2H), 3.69 (br, 2H), 4.72 (br, 1H), 5.31 (br, 1H), 7.39 (m, 10H), 11.73 (br, 1H); ¹³C NMR (DMSO-d₆) δ32.64, 36.95, 41.58, 51.16, 51.27, 121.55, 126.51, 128.20, 129,16, 139.17, 196.11; Anal. Calcd. For C₂₂H₂₅N₃S₂ HCl C 61.16; H 6.07; N 9.73; S 14.84; Found: C 61.33; H 6.27; N 9.38; S 14.52.

### Example 22

### 4-Dimethyl-piperazine-1-carbodithioc acid 3-cyano-3,3-diphenyl-propyl ester iodide (43) (Scheme 6)

Compound 22 was treated with CH₃l in ethyl acetate at room temperature overnight. The precipitated solid was washed with ethyl acetate to give compound 43 as a white solid. ¹H NMR (DMSO-d₆) δ2.85 (m, 2H), 3.21 (s, 3H), 3.25 (m, 2H), 3.33 (s, 3H), 3.55 (t, 4H, J=5.0 Hz), 4.26 (br, 2H), 4.53 (br, 2H), 7.37 (m, 2H), 7.44 (m, 8H); ¹³C NMR (DMSO-d₆)δ32.62, 36.97, 41.51, 41.56, 51.14, 51.22, 121.50, 126.46, 128.19, 129.16, 139.12, 196.45; MS (ESI) m/z 411 (M+1)⁺.

The synthesis described in Example 23 is outlined in Scheme 7.

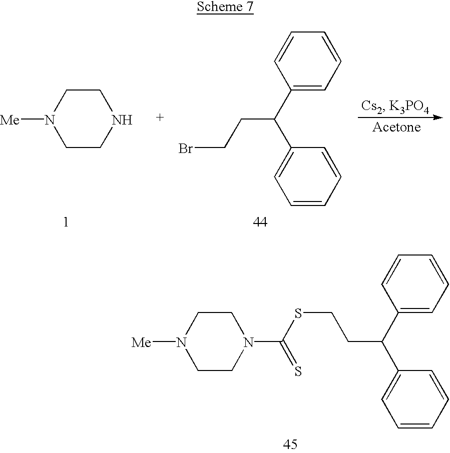

### Example 23

### Piperazine-1-carbodithioic acid 3-cyano-3,3-diphenyl-propyl ester (45) (Scheme 7)

Compound 45 was prepared as described above for the preparation of compound 22, this time employing compound 44 and compound 1. ¹H NMR (CDCl₃) δ1.72 (br, 1H), 2.83 (m, 2H), 2.93 (br, 4H), 3.39 (m, 2H), 3.87 (br, 2H), 4.29 (br, 2H), 7.30 (t, 2H), 7.38 (t, 4H), 7.46 (d, 4H); MS (ESI) m/z 382.4 (M+1)⁺.

The synthesis described in Example 24 is outlined in Scheme 8.

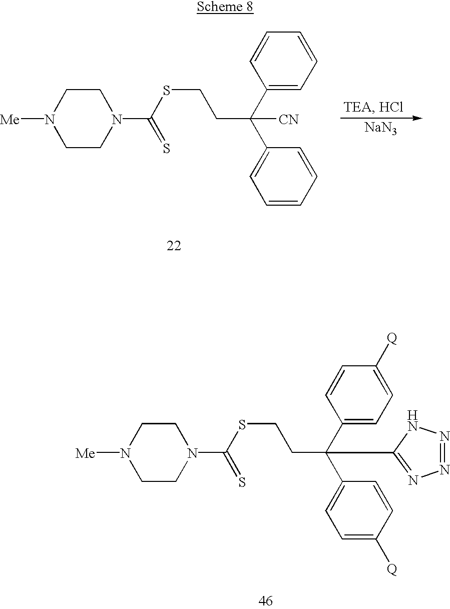

### Example 24

### 4-Methylpiperazine-1-carbodithioic acid 3-(5-tetrazolyl )-3,3-diphenyl-propyl ester (46) (Scheme 8)

A mixture of compound 22 (792 mg, 2 mmol) and triethylamine hydrochloride (390 mg, 6 mmol) in toluene (10 ml) was heated to reflux for 48 h. After cooling, the product was extracted with water (2×10 ml). The aqueous layer was neutralized to pH=7 with 5% HCl, and then extracted with ethyl acetate (4×10 ml). The organic phase was dried over anhydrous Na₂SO₄ and the solvent was evaporated. The residue was recrystallized from ethyl acetate to produce a yellow solid product 46. ¹H NMR (DMSO-d₆) δ2.25 (s, 3H), 2.45 (m, 4H), 2.50 (br, 1H), 3.03 (m, 4H), 3.88 (br, 2H), 4.17 (br, 2H), 7.22 (d, 4H, J=7.8 Hz), 7.28 (t, 2H, J=7.5 Hz), 7.34 (t, 4H, J=7.5 Hz); ¹³C NMR (DMSO-d₆) δ32.56, 38.04, 44.74, 49.21, 50.06, 51.26, 53.66, 127.00, 128.22, 128.24, 143.67, 161.90, 194.88; MS (ESI) m/z 439.8 (M+1)⁺.

The syntheses described in Examples 25-29 are outlined in Scheme 9.

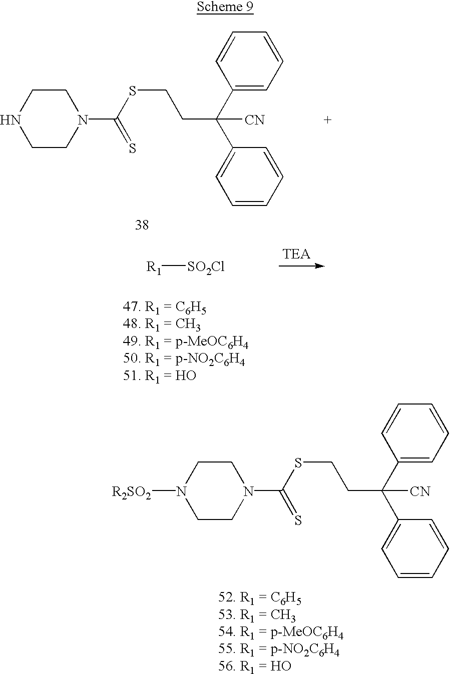

### Example 25

### 4-Phenylsulfonylpiperazine-1-carbodithioic acid 3-cyano-3,3-diphenyl-propyl ester (52) (Scheme 9)

To a solution of compound 38 and triethylamine in CH₂Cl₂ was added compound 47 dropwise at 0° C. The resulted solution was stirred at 0° C. for 2 h. The reaction solution was poured into 0.1 N HCl solution and extracted with CH₂Cl₂. The organic phase was washed with sodium bicarbonate solution and solvent was evaporated. The product was recrystallized to give the desired compound 52. ¹H NMR (CDCl₃) δ2.76 (m, 2H), 3.11 (br, 4H), 3.32 (m, 2H), 4.07 (br, 2H), 4.35 (br, 2H), 7.29 (d, 2H), 7.37 (t, 4H), 7.42 (d, 4H), 7.55 (t, 2H, J=7.5 Hz), 7.63 (t, 1H, J=7.5 Hz), 7.74 (d, 2H, J=7.5 Hz); ¹³C NMR (CDCl₃) δ32.77, 39.22, 45.68, 46.07, 49.79, 122.01, 127.14, 127.88, 128.34, 129.15, 129.57, 133.57, 135.46, 139.33, 197.14. MS (ESI) m/z 522.4 (M+1)⁺.

### Example 26

### 4-Methylsulfonylpiperazine-1-carbodithioic acid 3-cyano-3,3-diphenyl-propyl ester (53) (Scheme 9)

Compound 53 was synthesized as described above for the preparation of compound 52, this time employing compound 48 and compound 38. ¹H NMR (DMSO-d₆) δ2.85 (m, 2H), 2.92 (s, 3H), 3.24 (m, 4H), 3.52 (br, 2H), 4.12 (br, 2H), 4.35 (br, 2H), 7.36 (m, 2H), 7.45 (m, 8H); MS (ESI) m/z 482.5 (M+Na)⁺.

### Example 27

4-Methoxyphenylsulfonylpiperazine-1-carbodithioic acid 3-cyano-3,3-diphenyl-propyl ester (54) (Scheme 9)

Compound 54 was synthesized as described above for the preparation of compound 52, this time employing compound 49 and compound 38. ¹H NMR (CDCl₃) δ2.76 (m, 2H), 3.07 (br, 4H), 3.32 (m, 2H), 3.87 (s, 3H), 4.10 (br, 2H), 4.32 (br, 2H), 7.00 (d, 2H), 7.29 (t, 2H), 7.36 (m, 4H), 7.42 (d, 4H), 7.68 (d, 2H); MS (ESI) m/z 552.3 (M+1)⁺.

### Example 28

### 4-Nitrophenylsulfonylpiperazine-1-carbodithioic acid 3-cyano-3,3-diphenyl-propyl ester (55) (Scheme 9)

Compound 55 was synthesized as described above for the preparation of compound 52, this time employing compound 50 and compound 38. ¹H NMR (CDCl₃) δ2.76 (m, 2H), 3.16 (br, 4H), 3.32 (m, 2H), 4.10 (br, 2H), 4.32 (br, 2H), 7.30 (t, 2H), 7.35 (m, 4H), 7.41(d, 4H), 7.94 (d, 2H, J=9.0 Hz), 8.39(d, 2H, J=9.0 Hz); MS (ESI) m/z 589.9 (M +Na)⁺.

### Example 29

### 4-Sulfo-piperazine-1-carbodithioic acid 3-cyano-3,3-diphenyl-propyl ester (56) (Scheme 9)

Compound 56 is synthesized as described above for the preparation of compound 52, this time employing compound 51 and compound 38.

The synthesis described in Example 30 is outlined in Scheme 10.

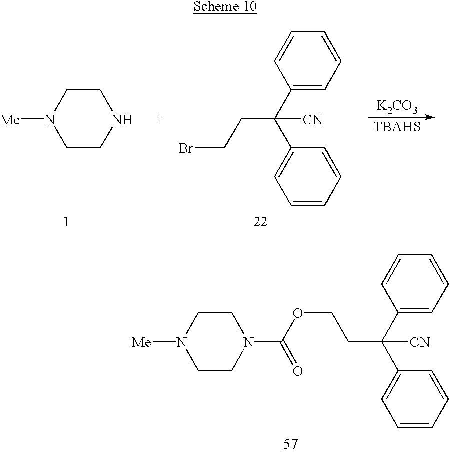

### Example 30

### 4-Methyl-piperazine-1-carboxylic acid 3-cyano-3,3-diphenyl-propyl ester (57) (Scheme 10)

A mixture of 1-Methylpiperazine (1) (0.1 g, 1 mmol), anhydrous potassium carbonate (2.2 g, 16 mmol), tetrabutyl-ammonium hydrogensulfate (TBAHS) (0.27 g, 0.8 mmol) and 2,2- diphenyl-4-bromobutyronitrile (19) (0.72 g, 2.4 mmol) in n-hetane (7 ml) was heated to reflux for 9 hr. After cooling, the mixture was filtered. The filtrate was washed with water and dried (Na₂SO₄). The solvent was evaporated and the residue was purified by column chromatography to give 60 mg (17%) of compound 57 as a pale yellow oil. ¹H NMR (CDCl₃) δ2.28 (s, 7H), 2.79 (t, 2H), 3.34-3.45 (d, 4H), 4.22 (t, 2H), 7.27-7.42 (m, 10H); Calcd. For C₂₁H₂₅N₃S₂, C 72.71; H 6.93; N 11.56; Found: C 72.91; H 7.14; N 11.11.

### Example 31

### Anticancer Activity of Hydrochloride Salt 42

Materials

Animals: Kuiming mice 18-22 g were provided by the animal department of Bejing Medical University. ICR mice were provided by Beijing Weitonglihua Experiemental Animal Technology, Inc. Each experiment used the same number of male mice and female mice.

Tumor cell line: Mouse sarcoma 180 (S180) was provided by the pharmaceutical group of National Key Laboratory of Natural and Mimic Medicine.

Compound 42 was prepared by the School of Pharmaceutical Sciences of Peking University. Cyclophosphoramide was purchased from Shanghai Hualian Pharmaceutical, Inc.

Procedure:

Tumor was removed from the mouse abdominal cavity. The tumor was diluted to 5×10⁶/ml and implanted to the left axilla subcutaneously (0.2 ml per mouse) under sterile conditions. Twenty four (24) hours after implantation, animals were divided into two groups (10 mice per group), either 42 or cyclophosphoramide was given orally to animals once a day for 10 days. Preliminary acute toxicity results are presented in Table 1.

TABLE 1
Preliminary Acute Toxicity

| Compound | Dose | Animal No. | Animal No. | No of |
|---|---|---|---|---|
| No. | G/kg | (start) | (close) | Death |
| 42 | 10 | 20 | 20 | 0 |

At day 12, the tumor was harvested and the average tumor weight of each group was calculated. Results, expressed in terms of % reduction in tumor mass, are presented in Table 2.

TABLE 2
Anticancer Activity of Compound 42

| | Dose | Animal No. | Inhibition |
|---|---|---|---|
| Compound No. | (mg/kg) | (start/close) | (%) |
| 42 | 500 | 10/9 | 80 |
| 42 | 100 | 10/10 | 74 |
| Cyclophosphoramide | 30 | 10/9 | 73 |

Conclusion: Compound 42 shows significant inhibition against S180. The compound also shows low toxicity.

While the invention has been described in detail with reference to certain preferred embodimens thereof, it will be understood that modifications and variations are within the spirit and scope of that which is described and claimed.

That which is claimed is:

1. A compound having the structure: 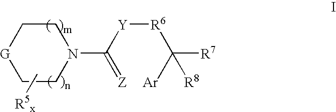
wherein:
is a divalent radical selected from CR¹R², O, S(O)_{0, 1, 2}, NR³ or N⁺R³R⁴ X⁻;
R¹ is H, OH, NO₂, COOH, CN, Cl, Br, I, keto (═O), thioketo (═S), imine (═NR, R is H, optionally substituted alkyl), an exocyclic double bond(═CH₂), —SO₃M or —OSO₃M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl, optionally substituted alkoxycarbonyl, alkylcarbonyloxy, optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino, optionally halogenated arylsulfonylamino, acyl, acyloxy, optionally substituted heterocyclic, a carbamate group, a sulfonamide group, sulfuryl, an amide group, or an optionally substituted ester group;
R² is absent when R¹ is keto, thioketo, imine, or an exocyclic double bond, or R² is H, OH, NO₂, COOH, CN, Cl, Br, I, —SO₃M or —OSO₃M, wherein M is H, Na, K, Zn, Ca or meglumine; CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl, optionally substituted alkoxycarbonyl, alkylcarbonyloxy, optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino, optionally halogenated arylsulfonylamino, acyl, acyloxy, optionally substituted heterocyclic, a carbamate group, a sulfonamide group, sulfuryl, an amide group, or an optionally substituted ester group;
R³ is H, optionally substituted hydrocarbyl, optionally substituted heteroaryl, optionally substituted aroyl, optionally substituted acyl, optionally substituted heterocyclic, hydroxyl, optionally substituted alkoxy, optionally substituted alkylthio, amino, optionally substituted alkylamino, optionally substituted dialkylamino, optionally substituted organosulfonyl, optionally substituted organosulfinyl, an optionally substituted alkylsulfonate ester group, optionally substituted alkylamino carbonyl, optionally substituted alkylamino thiocarbonyl, an optionally substituted alkyl ester group, an optionally substituted alkyl thioester group, an optionally substituted sulfonamide group, an optionally substituted alkyl carbonate group, an optionally substituted alkyl thiocarbonate group or an optionally substituted phosphonate group;
R⁴ is optionally present when X⁻ is present; when present, R⁴ is H, optionally substituted hydrocarbyl, optionally substituted heteroaryl or optionally substituted heterocyclic;
R⁵ is optionally present, and when present each R⁵ is independently selected from OH, NO₂, COOH, CN, Cl, Br, I, keto (═O), thioketo (═S), imine (═NR, R is H, optionally substituted alkyl), an exocyclic double bond(═CH₂), —SO₃M or —OSO₃M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl, optionally substituted alkoxycarbonyl, alkylcarbonyloxy, optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino, optionally halogenated arylsulfonylamino, acyl, acyloxy, optionally substituted heterocyclic, a carbamate group, a sulfonamide group, sulfuryl, an amide group, or an optionally substituted ester group;
R⁶ is optionally substituted alkylene, hydrocarbylene, optionally substituted aminoalkylene, or optionally substituted heterocyclic;
R⁷ is H, CN, COOH, OH, Br, Cl, I, optionally substituted aryl, optionally substituted heteroaryl, or R⁷ and Ar can cooperate to form a macrocycle;
R⁸ is H, OH, NO₂, COOH, tetrazolyl, C(O)NHOH, CN, Cl, Br, I, —SO₃M, or OSO₂M, wherein M is H, Na, K, Zn, Ca or meglumine, CSSH, COSH, CBr₂, CF₂, CF₃, CCl₂, CCl₃, optionally substituted hydrocarbyl, optionally substituted alkoxy, optionally substituted alkylthio, amino, monoalkylamino, dialkylamino, optionally substituted alkylcarbonyl; optionally substituted alkoxycarbonyl; alkylcarbonyloxy; optionally substituted heteroaryl, optionally substituted aroyl, optionally substituted arylcarbonyl, optionally substituted aryloxycarbonyl, optionally substituted arylcarbonyloxy, aralkyloxy, optionally substituted heteroarylcarbonyl, optionally substituted arylthio, carbamoyl, monoalkylcarbamoyl, di-alkylcarbamoyl, arylcarbamoyl, optionally substituted organosulfinyl, optionally substituted organosulfonyl, aryloxy, optionally halogenated alkylsulfonylamino or arylsulfonylamino, acyl, acyloxy, optionally substituted heterocyclic, a carbamate group, a sulfonamide group, sulfuryl, COOR⁹, COONR¹⁰R¹¹, wherein R⁹, R¹⁰ and R¹¹ are independently selected from C₁₋₁₀ alkyl;
Ar is optionally substituted aryl, optionally substituted heteroaryl, or Ar and R⁷ can cooperate to form a macrocycle;
X⁻ is present only when R⁴ is present; and when X⁻ is present, it is Cl⁻, Br⁻, I⁻, F⁻ nitrate, phosphate, sulfate, acetate, adipate, besylate, camyslate, citrate, edisylate, estolate, fumarate, gluceptate, gluconate, glucuronate, hippurate, hyclate, lactate, lactobionate, maleate, napsylate, oleate, pamoate, stearate, succinate, sulfosalicylate, tannate, tartrate, terephthalate, tosylate, or triethiodide;
m and n are independently integers from 0 up to 5;
x is an integer from 0 up to 4;
Y is a divalent radical selected from CR¹R², O or S; and
Z is O or S.

2. A compound according to claim 1 wherein:
G is N⁺R³R⁴ X⁻;
R³ is H, optionally substituted C₁₋₁₀ alkyl, or optionally substituted aryl;
R⁴ is present only if X is present, and when present R⁴ is H, or C₁₋₁₀ alkyl;
each R⁵, if present, is independently OH, NO₂, COOH, ═O, ═S, CN, Cl, Br, or optionally substituted lower alkyl;
R⁶ is an optionally substituted C₁₋₅ alkylene;
R⁷ is H, CN, COOH, optionally substituted aryl or optionally substituted heteroaryl;
R⁸ is H, OH, NO₂, COOH, tetrazolyl, CN, Cl, Br, I, or lower alkyl;
Ar is an optionally substituted aryl;
X⁻ is present only when R⁴ is present; and when X⁻ is present, it is Cl⁻, Br⁻, I⁻ or F⁻;
m and n are independently integers from 1 to 3;
x is an integer from 0 to 2; and
Y and Z are independently O or S.

3. A compound according to claim 1 wherein:
G is N⁺R³R⁴X⁻;
R³ is H, or an optionally substituted lower alkyl;
R⁴, if present, is H;
each R⁵ if present, is independently OH, CN, ═O, COOH or lower alkyl;
R⁶ is a C₁₋₃ alkylene;
R⁷ is H, COOH, CN, or optionally substituted aryl;
R⁸ is H, OH, COOH, CN or tetrazolyl;
Ar is an optionally substituted phenyl;
X⁻, if present, is Cl⁻;
m and n are both 1;
x is 0 or 1; and
Y and Z are S.

4. A compound according to claim 1 wherein:
G is N⁺R³R⁴X⁻;
R³ is an optionally substituted C₁₋₂ alkyl;
R⁴ is H;
R⁴ is not present;
R⁶ is ethylene;
R⁷ is an optionally substituted phenyl;
R⁸is COOH or CN;
Ar is an optionally substituted phenyl;
X⁻ is Cl⁻;
m and n are both 1;
x is 0; and
Y and Z are S.

5. A compound according to claim 1, wherein said compound has the structure referred to as compound 22, as follows:

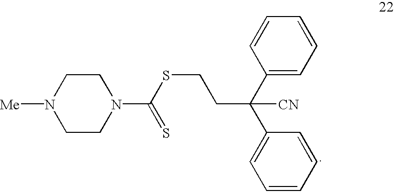

6. A compound according to claim 1, wherein said compound has the structure referred to as compound 42, as follows:

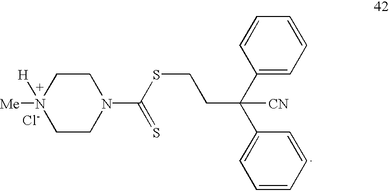

7. A method for the preparation of compounds according to claim 1, said method comprising:
a. contacting CHR⁷R⁸Ar (V) with Br—R⁶—Br (α,ω-Br,Br—R⁶) under conditions suitable to produce compound BrR⁶CR⁷R⁸Ar (VI), wherein R⁶, R⁷, R⁸ and Ar are as defined above
b. contacting compound VI with a 1-substituted (R³) piperazine (VII and carbon disulfide (CS₂) in the presence of a proton acceptor in a suitable solvent under conditions suitable to produce compound (VIII): 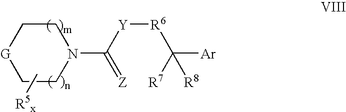
wherein
G, R³, R⁵, R⁶, R⁷, R⁸, Ar, m, n, x, Y and Z are as defined above.

8. A method according to claim 7, further comprising:
c. treating compound VII with alkyl halide and a suitable acid under conditions suitable to generate the corresponding salts (IX): 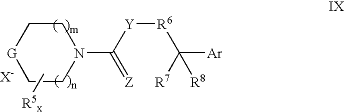
wherein
G, R³, R⁴, R⁵, R⁶, R⁷, R⁸, Ar, X⁻, m, n, x, Y and Z are as defined above:

9. A method according to claim 7, wherein the product is compound X, having the structure: 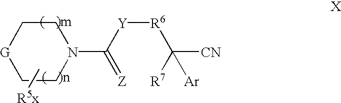
wherein
G, R⁵, R⁶, R⁷, Ar, m, n, x, Y and Z are as defined above.

10. A method according to claim 9, wherein compound X is subjected to conditions suitable to hydrolyze the cyanide group to a carboxylic acid, thereby producing compound XI, having the structure: 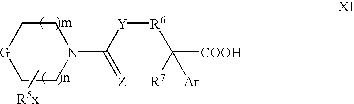
wherein
G, R⁵, R⁶, R⁷, Ar, m, n, x, Y and Z are as defined above.

11. A method according to claim 10, wherein compound XI is esterified to produce compound XII, having the structure: 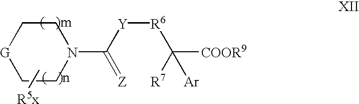
wherein
G, R⁵, R⁶, R⁷, R⁹, Ar, m, n, x, Y and Z are as defined above.

12. A method according to claim 9, wherein compound XI is contacted with an amine under conditions suitable to produce compound XIII, having the structure: 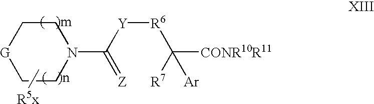
wherein
G, R⁵, R⁶, R⁷, R¹⁰, R¹¹, Ar, m, n, x, Y and Z are as defined above.

13. A method according to claim 9, wherein compound X is subjected to conditions suitable to produce compound XIV, having the structure: 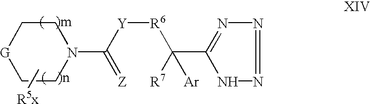
wherein
G, R⁵, R⁶, R⁷, Ar, m, n, x, Y and Z are as defined above.

14. A method for the treatment of a pathological condition in a subject in need thereof, said method comprising administering an effective amount of a compound according to claim 1 to said subject.

15. The method according to claim 14 wherein said pathological condition is septic shock, hemorrhagic shock, anaphylactic shock, toxic shock syndrome, ischemia, cerebral ischemia, administration of cytokines, overexpression of cytokines, ulcers, inflammatory bowel disease, diabetes, arthritis, asthma, Alzheimer's disease, Parkinson's disease, multiple sclerosis, cirrhosis, allograft rejection, encephalomyelitis, meningitis, pancreatitis, vasculitis, lymphocytic choriomeningitis, glomerulonephritis, uveitis, ileitis, inflammation, burn, infection, hemodialysis, chronic fatigue syndrome, stroke, cancers, cardiopulmonary bypass, ischemic/reperfusion injury, gastritis, adult respiratory distress syndrome, cachexia, myocarditis, autoimmune disorder, eczema, psoriasis, heart failure, heart disease, atherosclerosis, dermatitis, urticaria, systemic lupus erythematosus, AIDS, AIDS dementia, chronic neurodegenerative disease, pain, priapism, cystic fibrosis, amyotrophic lateral sclerosis, schizophrenia, depression, premenstrual syndrome, anxiety, addiction, migraine, headache, Huntington's disease, epilepsy, neurodegenerative disorders, gastrointestinal motility disorders, obesity, hyperphagia, solid tumors, malaria, hematologic cancers, myelofibrousis, lung injury, graft-versus-host disease, head injury, CNS trauma, hepatitis, renal failure, liver disease, drug-induced lung injury, myasthenia gravis (MG), ophthalmic disease, post-angioplasty, restenosis, angina, or coronary artery disease.

16. A method according to claim 15 wherein said pathological condition is cancer and/or tumor.

17. A formulation comprising a compound according to claim 1 in a pharmaceutically acceptable carrier therefor.

18. A formulation according to claim 17 wherein said pharmaceutically acceptable carrier is a solid, solution, dispersion, micelle or liposome.

19. A formulation according to claim 17 wherein said pharmaceutically acceptable carrier further comprises an enteric coating.

20. An antibacterial formulation comprising a compound according to claim 1 in a suitable carrier therefor.

21. A method for controlling bacterial contamination, said method comprising applying a formulation according to claim 20 to any surface suspected of bacterial contamination.

22. An insecticidal formulation comprising a compound according to claim 1 in a suitable carrier therefor.

23. A method for controlling insect activity, said method comprising applying a formulation according to claim 22 to any surface where insect control is desired.
